(19) United States
(12) Patent Application Publication
(10) Pub. No.: US 2012/0190923 A1
(43) Pub. Date: Jul. 26, 2012

(54) ENDOSCOPE

(75) Inventors: Martin Kunz, Munchen (DE); Anton Schick, Velden (DE); Michael Stockmann, Bruckmuhl (DE)

(73) Assignee: SIEMENS AKTIENGESELLSCHAFT, Munchen (DE)

(21) Appl. No.: 13/498,984

(22) PCT Filed: Sep. 29, 2010
(86) PCT No.: PCT/EP2010/064428

§ 371 Date: Mar. 29, 2012

(30) Foreign Application Priority Data
Sep. 30, 2009 (DE) 10 2009 043 523.9

(51) Int. Cl. A61B 1/04

(52) U.S. Cl. 600/109

(57) ABSTRACT

An endoscope measures the topography of a surface. The endoscope contains a projection unit and an imaging unit. The projection unit and the imaging unit are arranged successively in relation to an axis of the endoscope. The configuration of the projection unit and the imaging unit arranged axially behind one another on the axis permits a significantly smaller endoscope configuration.

The invention relates to an endoscope for measuring the topography of a surface as claimed in the preamble of claim 1 and a method for measuring the topography of a surface as claimed in claim 20.

Conventional and well researched techniques for measuring three-dimensional geometries are frequently based on active triangulation. However, in a narrow environment such as, for example, in the human auditory canal or in drill holes, it is increasingly difficult to implement triangulation as such. In particular in the field of measuring endoscopy, it is not easy to position the spatial arrangement of transmit and receive unit or projection and imaging unit under the corresponding angles. In addition, as a rule it is not possible to include longer or larger hollow spaces in an image. This means it is necessary to measure spatially overlapping regions three-dimensionally in temporal succession in order to then combine them by data processing means to form a 3D configuration (3D data sticking). Here, the larger the overlapping areas, the more precisely the interconnection of individual images in 3D space can take place. This also requires the individual images per se to have a fixed relationship to each other at as many measuring points as possible.

The object underlying the invention is to provide an endoscope for measuring surface topographies requiring less mounting space than the prior art and which is able, for example, when using active triangulation, to cover larger measuring areas in just one measuring sequence.

The object is achieved by an endoscope with the features of claim 1 and by a method with the features of claim 20. The endoscope according to the invention for measuring the topography of a surface comprises a projection unit and an imaging unit. The endoscope is characterized by the fact that the projection unit and the imaging unit are arranged behind one another in relation to an axis of the endoscope.

This arrangement of projection unit and imaging unit (also receive unit) arranged axially behind one another on an axis (axis of the endoscope) make it possible, with a suitable design of the projection lens or of the receive lens, to achieve ideal overlapping of the projection area and the imaging area with a narrow hollow space. This arrangement according to the invention of the projection unit and imaging unit makes much better use of the available mounting space in an endoscope, which permits a significantly smaller endoscope design.

With the axial arrangement of the projection unit and the imaging unit, the imaging unit can in principle be aligned in the same viewing direction in relation to the axis of the endoscope as the projection unit. With suitable imaging optics, the imaging unit can also be arranged opposite to the viewing direction of the projection unit. A face to face arrangement of the projection unit and the imaging unit of this kind only differs in the embodiment of the imaging optics, but in principle the arrangement provides the same advantages for measuring 3D surfaces in a narrow space. The term “viewing direction” should be understood to mean the direction along the axis of the endoscope in which the endoscope is guided.

An arrangement of this kind is in particular suitable for the use of active triangulation. The space-saving arrangement of the projection unit and the imaging unit creates advantageous possibilities for the design of the measuring unit, which will be dealt with in more detail below. Moreover, a much higher number of color-coded patterns are available for so-called color-coded triangulation, thus enabling more precise measurement of the topography of the surface.

In an advantageous embodiment of the invention, projection rays from the projection unit extend radially laterally past the imaging unit and emerge laterally from an endoscope wall. The endoscope outer material is correspondingly optically transparent, wherein, as rule, the material used is glass or transparent plastic, such as Plexiglas. The radial lateral emergence of the projection rays represents an embodiment which allows the projection rays to emerge from the endoscope and land on the surface without being impeded by the imaging unit.

In a further advantageous embodiment of the invention, the light supply to the projection unit takes place via an optical waveguide or optical waveguide bundle. The light can be fed into the optical waveguide by an LED, for example. The use of a waveguide also saves space and moreover, in the area of the endoscope measurement, no heat will be emitted by a lighting means, which can also be detrimental in medical applications.

To measure the topography by means of triangulation, it is expedient for a projection structure with color coding to be provided between the light supply and projection optics of the projection unit. This projection structure can be embodied as a radially symmetrical structure, in particular if the lighting unit is embodied in the form of an optical waveguide with a round cross section. The projection structure is expediently embodied in the form of a slide.

Here, the slide comprises, at least in an external area, a plurality of concentric colored rings. These colored rings serve as color coding—the more colored rings can be attached to the slide or to the projection structure, the greater the measuring area of individual measurements and, as a result, it is possible to dispense with so-called feature tracking.

In a preferred embodiment, the projection structure, in the special case the slide, is arranged directly before the optical waveguide, wherein the projection rays extend perpendicularly through the projection structure.

In the case of a projector unit which is telecentric in relation to the slide, ray bundles emitted by the slide are guided through the projection optics. The respective main beams of the bundles extend perpendicularly to the slide and intersect in the pupil of the projection optics. From there, the main beams (which are parts of the projection rays) diverge and emerge from the endoscope wall and then land on the surface to be measured. A telecentric projection unit of this kind also saves mounting space since it is possible to dispense with so-called collimation optics.

The imaging unit of the endoscope comprises an imaging medium, which is preferably embodied in the form of a sensor chip of a digital camera.

The imaging unit also comprises imaging optics, which can cover a field of view, the size of which is adapted to the projection area. Here, the area of intersection of the field of view and projection area defines the measuring area.

In an advantageous embodiment of the invention, the imaging optics comprise a convex mirror and a planar mirror, wherein the convex mirror is convexly arched in the direction of the planar mirror. The convex mirror serves inter alia to deflect the imaging rays (imaging rays are projection rays reflected at the surface) onto the planar mirror. The planar mirror in turn deflects the imaging rays once again so that they extend through a central opening in the convex mirror. Here, the imaging medium is arranged behind the convex mirror in relation to the viewing direction of the endoscope. The imaging rays are deflected through the central opening in the convex mirror directly or indirectly onto the imaging medium. This measure enables the field of view of the imaging unit to be embodied as very large. A field-of-view angle of more than 180° is possible. In this described embodiment, the imaging medium is arranged behind the imaging optics in relation to a viewing direction of the axis of the endoscope. Hence, the imaging unit comprises a viewing direction corresponding to the viewing direction of the endoscope.

However, it is also possible to invert the viewing direction of the imaging unit so that it is arranged opposite to the viewing direction of the endoscope. In this case, the imaging medium is disposed behind the imaging optics of the imaging unit in relation to the viewing direction of the endoscope.

In a further embodiment of the invention, it is expedient for the planar mirror also to comprise a, preferably central, opening which serves to allow the passage of light rays. Here, these are light rays extending opposite to the viewing direction of the endoscope. This makes it possible for objects or surfaces in the viewing direction of the endoscope to be received and pass through the opening of the planar mirror and through the opening of the convex mirror and land in an area of the imaging medium near the center where they can be detected. An additional lens arrangement in the area of the opening can serve to improve the imaging quality and adjust the magnification. This measure enables the endoscope to be used both as a camera endoscope and as a measuring endoscope.

A method as claimed in claim 2 is also part of the invention. The method according to the invention serves to measure the topography of a surface by means of an endoscope as claimed in any one of claims 1 to 19.

It is characterized by the fact that projection rays are emitted by a projection unit, the projection rays emerge laterally and radially from an endoscope wall, the projection rays are reflected by a surface to be measured and depicted by an imaging unit in the endoscope in a planar way on an imaging medium, wherein the imaging unit is arranged before the projection unit in relation to an axis of the endoscope.

Further advantageous embodiments of the invention will be explained in more detail with reference to the figures. Here, features with the same designation but in different embodiments are provided with the same reference characters.

The figures show:

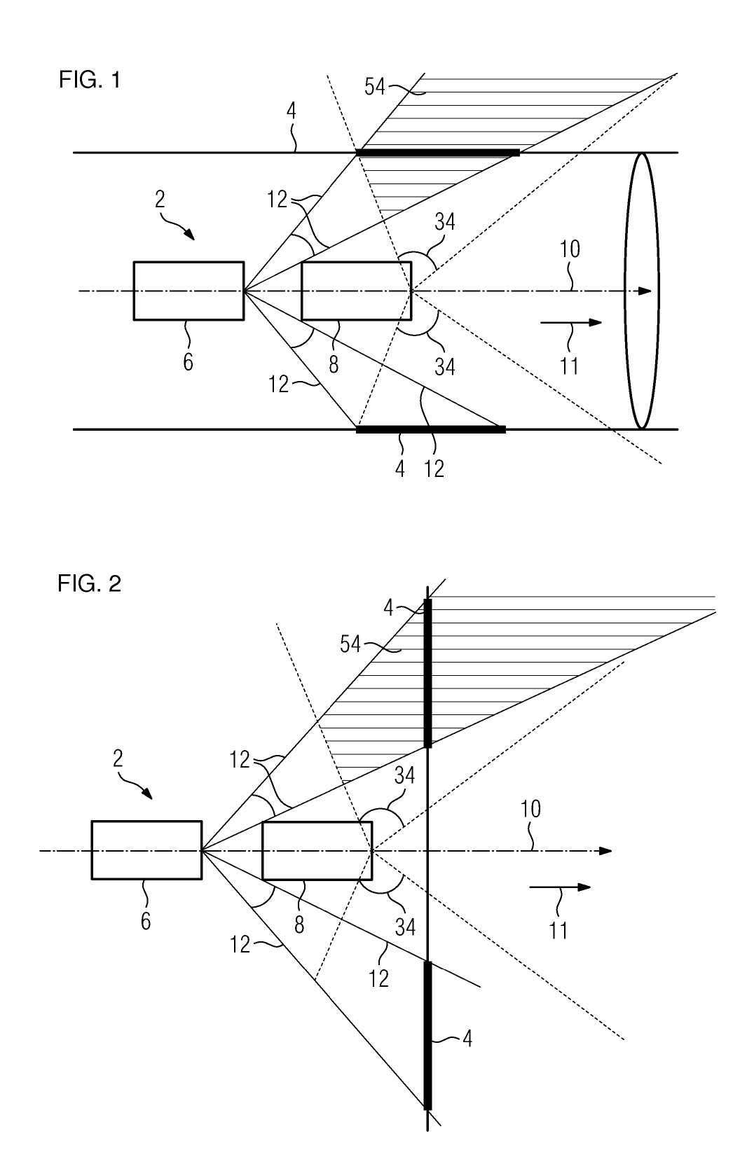

FIG. 1 a schematic representation of a measuring endoscope with a projection unit and an imaging unit for measuring a surface parallel to the axis of the endoscope,

FIG. 2 an endoscope with the structure shown in FIG. 1 for measuring a surface perpendicular to the axis of the endoscope,

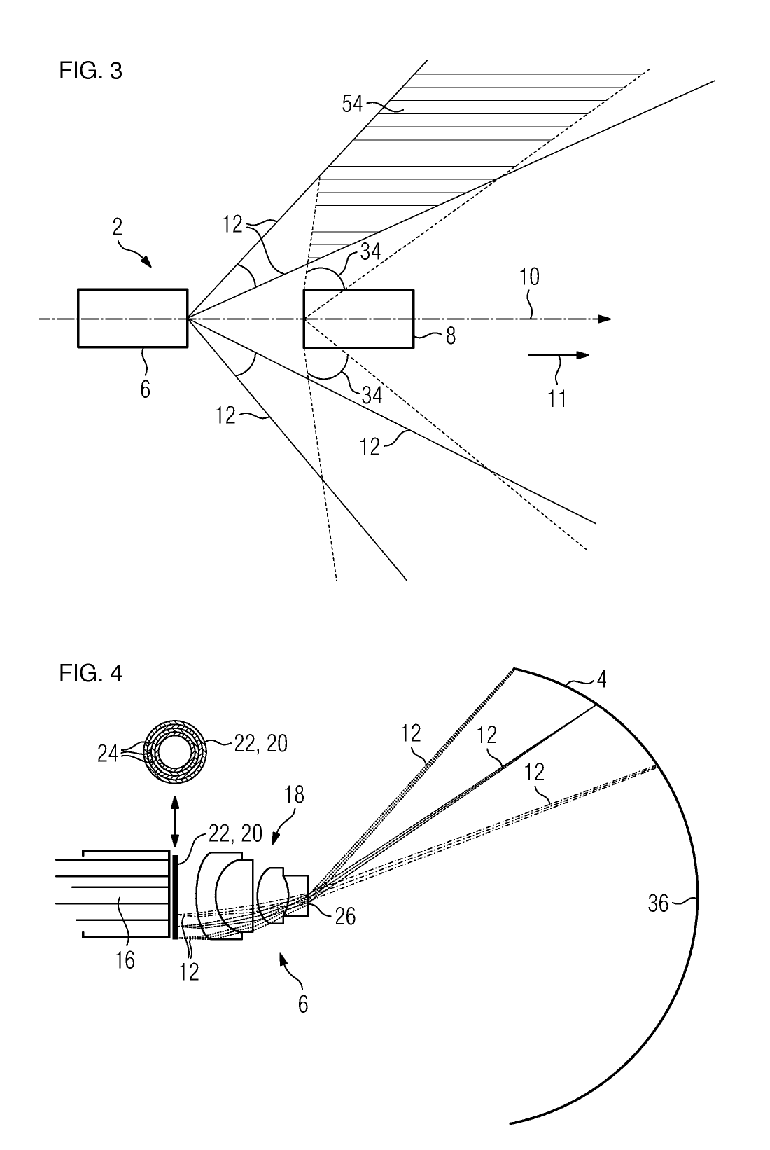

FIG. 3 a schematic representation of an endoscope, wherein the imaging unit and the projection unit have opposite viewing directions,

FIG. 4 a schematic representation of the projection unit with a ray trace,

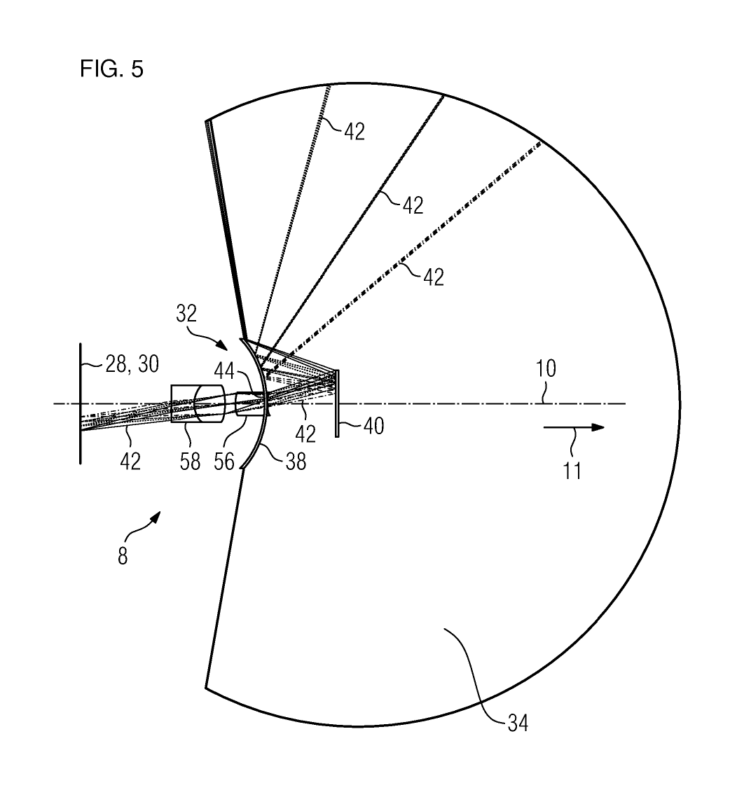

FIG. 5 a schematic representation of the ray trace of the imaging unit,

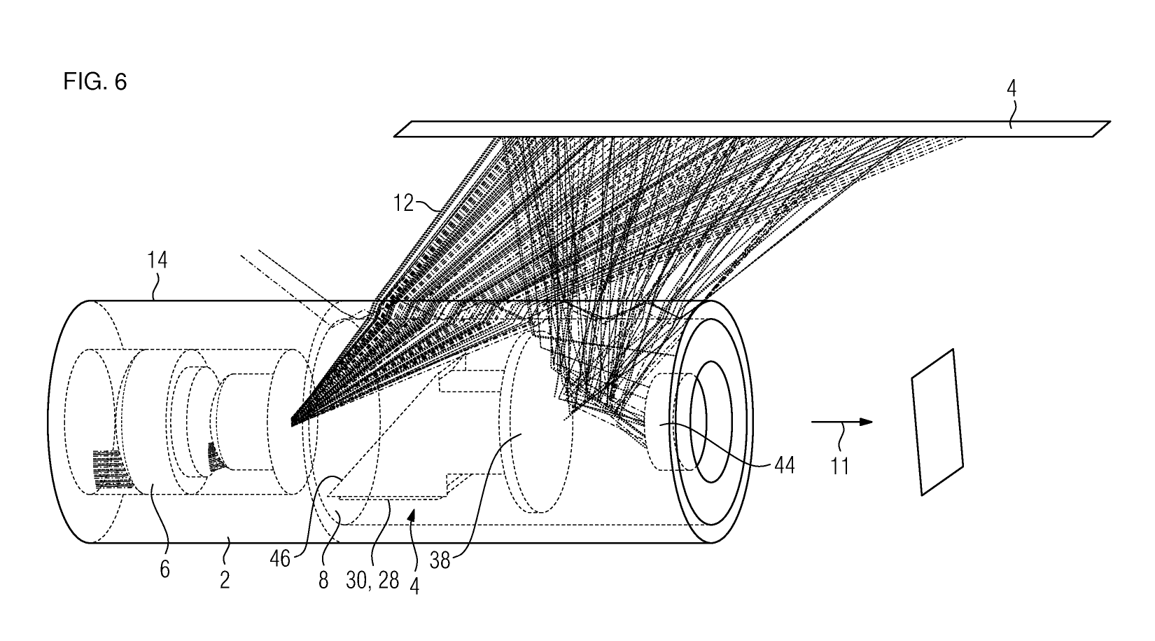

FIG. 6 a schematic three-dimensional transparent representation of an endoscope with a ray trace according to FIG. 1 or 2,

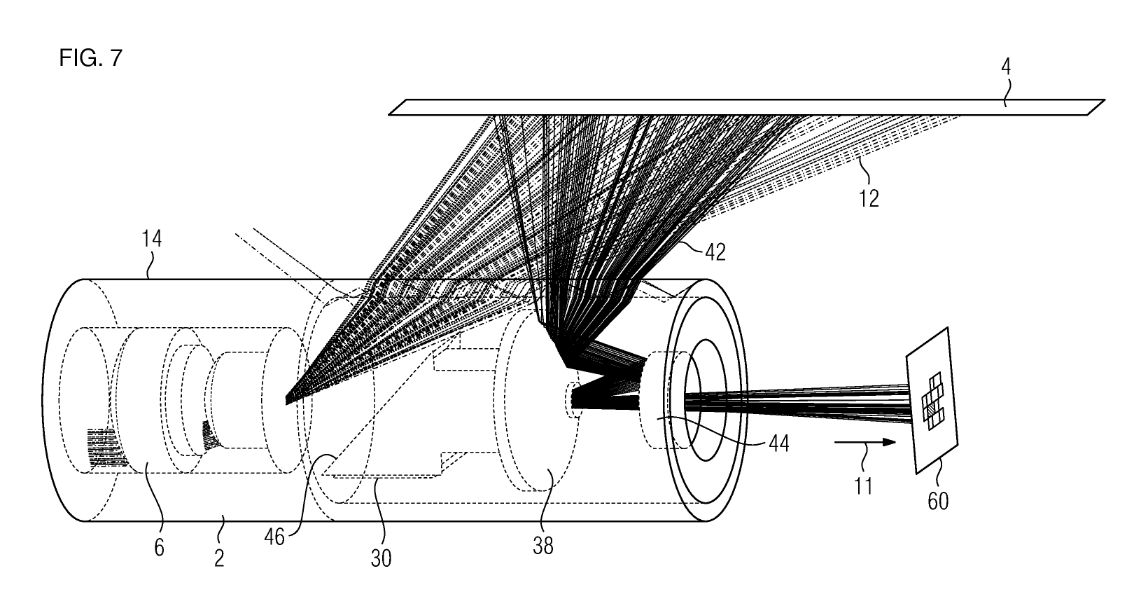

FIG. 7 a three-dimensional transparent representation of an endoscope as shown in FIG. 6 but with the additional reception of rays from the viewing direction of the endoscope,

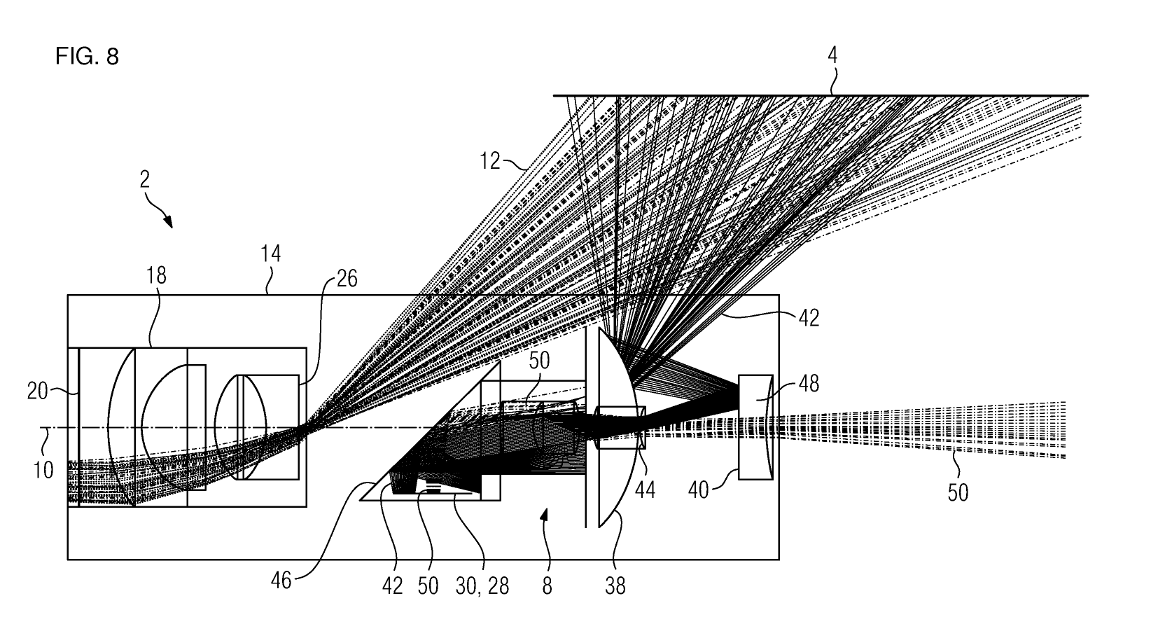

FIG. 8 a schematic representation of the ray trace of the endoscope shown in FIG. 7 and

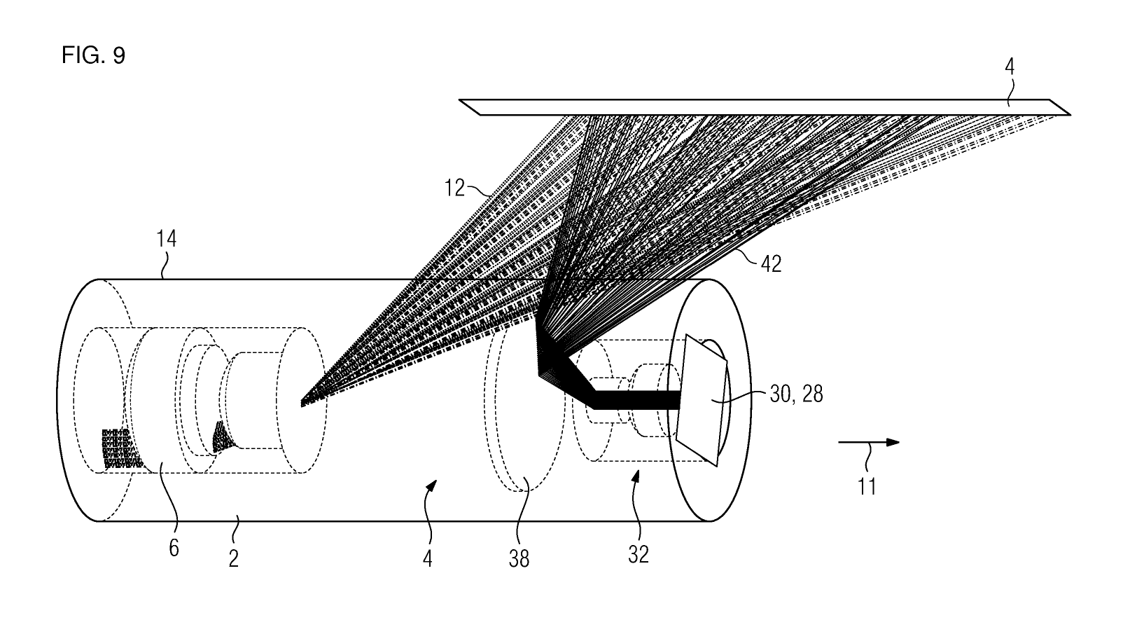

FIG. 9 a three-dimensional transparent representation of an endoscope with an embodiment of a projector unit and imaging unit according to FIG. 3.

FIGS. 1 and 2 show an embodiment of a 3D measuring endoscope with a projector unit 6 and an imaging unit 8 arranged behind one another on an axis of the endoscope 10. The endoscope 2, the outer wall 14 of which (see for example FIG. 6) is not explicitly shown in these figures, serves for measuring a surface 4. Here, as shown in FIG. 1, the surface 4 can be a channel, for example an auditory canal of a human ear or a drill hole which is why the wall 4 is shown as cylindrical in the schematic representation in FIG. 1. Unlike the case in FIG. 2, this shows, how the same endoscope 2 is used to measure the topography of a rather perpendicular wall 4. In reality, the wall 4 to be measured obviously has a complex form, the straight lines, which are designated 4 in FIGS. 1 and 2, only serve to provide a schematic graphical illustration.

The method of triangulation is used to measure the topography of the surface 4. To this end, projection rays 12, which may have different color spectra, are emitted by the projection unit 6. These projection rays 12 land on the surface 4 and are reflected there. Due to suitable imaging optics, the imaging unit 8 in turn has a field of view 34, which is illustrated in both FIGS. 1 and 2 by the dashed lines. Here, it should be noted that in reality both the projection rays 12 and the field of view 34, which are shown two-dimensionally in FIGS. 1 and 2, extend three-dimensionally and rotationally symmetrically.

The area, which is encompassed by both the projection rays 12 and the field of view 34, that is the area in which the projection rays 12 and the field of view 34 intersect, is called the measuring area 5 and is shown hatched in FIGS. 1 and 2.

A measurement using a triangulation method can only take place in the area in which the projection rays 12 and field of view 34 intersect. The larger the measuring area 54, the larger the area that can be covered in one measurement. In particular in narrow hollow spaces, with known methods, it is frequently difficult to embody the field of the projection rays and the field of view in such a way that a sufficiently large measuring area 54 is formed.

The described row arrangement of the projection unit 6 and the imaging unit 8 on the axis of the endoscope 10 enables the ray trace described in FIGS. 1 and 2 to be achieved. Here, it is expedient for the projection rays 12 to be diverted radially and laterally through suitable projection optics past the imaging unit 8. The projection rays emerge from a wall (not shown here) (see for example reference number 14 in FIG. 6) and land on the surface 4 to be measured. The imaging unit 8, the viewing direction of which is identical to the viewing direction 11 of the endoscope (FIG. 1 toward the right), in turn comprises an advantageous embodiment of a very large field of view 34 (field of view). The field of view 34 of the imaging unit 8 can be more than 180°. It is expedient for the field of view 34 in principle to have a larger angle than the maximum angle enclosed by projection rays. The embodiment of imaging optics which provides a field of view 34 of this kind will be dealt with further below.

First, at this point, there will be a discussion of FIG. 3, which also shows a measuring endoscope 2 having the same series construction (or row construction) of the projection unit 6 and imaging unit 8 on an axis of the endoscope 10, the projection unit 6 corresponding to the projection unit 6 in FIGS. 1 and 2 and the ray trace of the projection rays 12. The only difference from FIGS. 1 and 2 consists in the fact that the imaging unit 8 is virtually rotated by 180° and is embodied in the field of view 34 in such a way that the viewing direction of the imaging unit 8 is opposite to the viewing direction 11 of the endoscope 2. The triangulation method measurement is performed similarly to that in FIGS. 1 and 2. Once again a measuring area 54 forms in the area of intersection between the projection rays 12 and the field of view 34. This arrangement in FIG. 3 can, for example, be used if additional visualization in the viewing direction 11 of the endoscope 2 is necessary. In this case, an additional camera lens with an image sensor can be accommodated at the end of the endoscope 2.

The following will describe the projection unit 6 and projection optics 18 in more detail with reference to FIG. 4. The projection unit 6 comprises a light source, which is here embodied in an advantageous way in the form of a waveguide or waveguide bundle 16. Upstream of the light source, there is a projection structure 20, which is here embodied as a slide 22. The slide 22 in FIG. 4 comprises a plurality of concentric colored rings 24. In addition to the cross section through the slide 22, FIG. 4 also shows a top view of the slide 22 which serves better to illustrate the arrangement of the concentric colored rings 24. The projection structure 20 can in principle also be embodied in the form of a colored line structure or a line structure embodied in some other way. The embodiment shown here is the so-called color-coded triangulation method, wherein the colored rings 24 (usually between 15 and 25 pieces, preferably about 20 pieces) form a color-coded ring pattern.

The projection rays 12, which come from the optical waveguide 16 and which, in this example are emitted by an LED (not shown here), extend virtually perpendicularly through the slide 22, are deflected by suitable projection optics 18 and meet each other in a pupil 26 in such a way that in each case main beams meet in the pupil 26 in a virtually punctiform manner. This is referred to as a slide-side telecentric projector unit.

Further on, the individual projection rays 12 separate according to their color and land as a color pattern on the surface 4 to be measured. The surface 4 to be measured is now shown in FIG. 4 as a circular field. The fanning out of the projection rays 12 produces a so-called projection area 36.

The irregular topography of the surface 4 (which is not shown here) causes the projection rays 12, which formerly extended parallel when passing through the slide 22, to land at different distances from the projection lens on the surface 4. Seen from another viewing direction, the projection image reflected on the surface 4 appears distorted and is depicted by imaging optics to be described below on an imaging medium 28, wherein a suitable evaluation method can be used to calculate the topography of the surface 4 from an evaluation of the color transitions and the distortion of the color lines.

There now follows a description of an advantageous imaging unit 8 with advantageous imaging optics 32. The projection rays 12 reflected at the surface 4 are described in the following as imaging rays 42. The imaging rays 42 land on a convex mirror 38, which is convexly arched in the viewing direction 11 of the endoscope. The convex mirror 38 reflects the imaging rays 42 in the viewing direction 11 of the endoscope 2 onto a further planar mirror 40, which in turn reflects the imaging rays a further time. This second reflection of the imaging rays 42 is directed in such a way that the reflected rays 42 are diverted through an opening 44 in the convex mirror 38.

This opening 44, which is in particular arranged centrally in the mirror 38, contains a lens 56 via which the rays 42 extend further through an achromatic lens 58 and finally land on an imaging medium 28, which in this example is embodied as a sensor chip 30, such as those also used, for example, in digital cameras. In principle, it is possible, to arrange a further prism 46 between the achromatic lens 58 and the sensor chip 30, as shown in FIG. 7 and also in FIG. 6, which enables the sensor chip 30 to be transposed with respect to its position in relation to the axis of the endoscope 10. It can be expedient to arrange the sensor chip 30 parallel to the axis of the endoscope. This means that a surface normal of the sensor chip 30 extends perpendicularly, or at least not parallel to, the axis of the endoscope 10.

For a better illustration of the formerly abstract representation of the ray traces in the endoscope 2, FIG. 6 shows a three-dimensional transparent representation of an endoscope 2 in an end area. This embodiment in FIG. 6 corresponds to the ray traces shown in FIGS. 1 and 2. In this representation, for better clarity, the ray traces of the imaging rays 42 are not shown completely (for this, see FIG. 8). In FIG. 6, once again, only the ray traces are depicted schematically, wherein attention is drawn to the representation of the physical units of the endoscope 2, namely the projection unit 6, and the imaging unit 8. The diameter of the endoscope is preferably between 3 mm and 5 mm. The projection unit is normally about 10 mm long.

The projection unit 6 emits the projection rays 12 through the endoscope wall 14 radially toward the outside. Once again, the ray direction shown here is only for greater clarity. In reality, the projection rays emerge rotationally symmetrically from the endoscope 2. At the surface 4, the projection rays 12 are reflected and received by the imaging unit 8. The imaging unit 8 is arranged on the axis of the endoscope 10 before the projection unit 6 in the viewing direction 11. The preposition “before” indicates that the imaging unit 8 is arranged in the direction of the arrow 11 on the axis of the endoscope in relation to the projection unit 6. The preposition “before” is used with this meaning in the following. The preposition is used for an arrangement of a named subject opposite to the arrow direction.

The imaging rays 42 (not shown here, see FIG. 8) are, as already described in FIG. 5, diverted via the convex mirror 38 and the planar mirror 34 onto the sensor chip 30, wherein, in this embodiment, they are also diverted via a prism 46 onto the sensor chip 30.

An, in principle, identical arrangement to that in FIG. 6 is shown in FIG. 7. However, the embodiment illustrated in FIG. 7 also makes it possible for the endoscope to receive further objects 60 lying in the viewing direction 11 of the endoscope.

The manner in which this additional function of the endoscope 2 is embodied according to FIG. 7 is shown schematically in FIG. 8. In relation to measuring endoscopy, FIG. 8 comprises the same ray trace of the projection rays 12 and the imaging rays 42 as that shown in FIGS. 1, 2, 4, 5, 6 and 7. The projection unit 18 projects colored projection rays 12 via projection optics 18 radially past the imaging unit 8 onto the surface 4. The surface 4 reflects the projection rays 12 in the form of imaging rays 42, which are received and diverted via the convex mirror 38 and pass via the planar mirror 40 through an opening 44 in the convex mirror 38 to land on the sensor chip 30.

As can be seen in FIG. 4, the annular structure of the slide 22 has a concentric opening in the center. Consequently, the projection rays 12 to be analyzed only pass through the outer area of the slide 22. The central area of the slide 22 is not used for the projection or for the imaging. This also means that the imaging on the sensor chip 30 also only takes place in the outer area of the sensor chip. The central area of the sensor chip is not illuminated by the ray trace of the projection rays 12 and the imaging rays 42.

Hence, the central area of the sensor chip 30 can be used for a further function. For this reason, it has been found to be expedient also to provide the planar mirror 40 with a central opening 48 to allow the passage of the light rays 50 which are reflected by objects 60 and are arranged in the viewing direction 11 of the endoscope 2. These light rays 50 pass through the opening 48 of the planar mirror and through the opening 44 of the convex mirror 38 and then land in the central area of the sensor chip. Hence this central area of the sensor chip 30 can be used for the visualization of the objects 60 lying in the viewing direction 11 of the endoscope.

Hence the endoscope 2 has a dual function as a camera and as a measuring endoscope for the determination of the surrounding topography. This advantageous embodiment according to FIG. 8 enables the operator during the control of the endoscope simultaneously to identify what is taking place before his endoscope so that reliable guidance of the endoscope is enabled. Generally, the scattered light of the projection rays is sufficient to illuminate objects 60 before the endoscope. For an otoscope function of the endoscope, the image rate could be reduced to up to 2 Hz. If the light should be too low for the observation of the objects 60, an additional lighting unit can be attached in the front endoscope area.

Usually, to receive the imaging rays 42, the sensor chip is illuminated with a frequency of 10 Hz. Here, the shutter opening time is about 10 ms. This means that, at an illumination frequency of 10 Hz, there is a pause of 90 ms between the shutter openings. During this time, the sensor chip recordings are evaluated by calculation software. (The shutter opening time is the time in which the imaging rays 42 landing on the sensor chip are measured.)

There now follows a description of FIG. 9, which shows a three-dimensional, transparent representation of an endoscope 2 according to FIG. 3. As described above, the embodiment of the endoscope 2 according to FIG. 3 only differs from FIGS. 1 and 2 in that the imaging unit 8 is rotated with respect to its viewing direction by 180° in relation to the viewing direction 11 of the endoscope. In practice, this means that the imaging optics 32 substantially have the same embodiment, but with an embodiment of this kind, the imaging medium 28, in particular the sensor chip 30, is disposed before the imaging optics 32 in the viewing direction 11 of the endoscope 2. (Contrary to this, the imaging medium 28 lies behind the imaging optics 32 in relation to the viewing direction 11 when, as shown in the examples in FIGS. 1 and 2, the imaging unit 8 has the same viewing direction as the viewing direction 11 of the endoscope.) The imaging unit in FIG. 9 also comprises a convex mirror 38, which serves to provide a field of view of more than 180° C. The mirror 38 diverts the imaging rays 42 through imaging optics 32 to the sensor chip 30 where they are detected.

It is also expedient to arrange a further reception unit (not shown) in a measuring endoscope according to FIG. 9 before the imaging unit, which optionally comprises a separate sensor chip and separate optics and which is in particular used for the optical detection of objects lying before the endoscope. Hence, the endoscope has a measuring function for measuring the surface topography and a viewing function enabling the user to see well into the area to be measured and guide the endoscope.

The arrangement of the measuring endoscope 2 described can, in principle, be used for all measurements in narrow hollow spaces. A particularly advantageous use of the endoscope 2 is depicted in the form of an otoscope suitable for measuring purposes, which is introduced into an ear and is used to measure the auditory canal or (see FIG. 2) to measure the auricular muscle, for example to produce a suitable hearing aid. Here, the above-described so-called color-coded triangulation has the advantage that, the projection of an encoded color pattern is sufficient with only one image of the receive unit (imaging unit 8) for the calculation of the 3D shape of an object. This means that it is possible to use simple projection in analogy to slide projection and no additional change to the projection structure is required, unlike the case, for example, with so-called phase triangulation. This also has the advantage that a doctor can perform free-hand scanning with virtually no shaking.

Other applications of the endoscope 2 could lie within a technical field. The use of a space-saving endoscope 2 of this kind is expedient if, for example, drill holes or other cavities have to be measured precisely for purposes of quality assurance. For example, very high requirements are placed on the topography of rivet holes used for the riveting of aircraft components. An endoscope according to the invention of this kind enables high-precision topography measurements to be taken in very narrow holes.

1-20. (canceled)

21. An endoscope for measuring a topography of a surface, the endoscope comprising:
a projection unit; and
an imaging unit, said projection unit and said imaging unit disposed behind one another in relation to an axis of the endoscope, said imaging unit disposed on said axis of the endoscope in a viewing direction of the endoscope before said projection unit.

22. The endoscope according to claim 21, wherein a measurement of the topography is performed by means of active triangulation.

23. The endoscope according to claim 21, wherein projection rays from said projection unit extend radially and laterally past said imaging unit.

24. The endoscope according to claim 23, further comprising an endoscope wall, the projection rays emerge laterally from said endoscope wall.

25. The endoscope according to claim 21, further comprising a light supply for said projection unit, said light supply is an optical waveguide.

26. The endoscope according to claim 25,
wherein said projection unit has projection optics; and
further comprising a projection structure with color coding disposed between said light supply and said projection optics of said projection unit.

27. The endoscope according to claim 26, wherein said projection structure has a radially symmetrical structure.

28. The endoscope according to claim 26, wherein said projection structure is embodied in a form of a slide.

29. The endoscope according to claim 28, wherein said slide is embodied with color coding containing concentric colored rings.

30. The endoscope according to claim 29, wherein said projection structure is disposed directly before said optical waveguide and projection rays emitted from said projection unit extend telecentrically between said projection structure and said projection optics.

31. The endoscope according to claim 30, wherein said projection optics have a pupil in a region of which ray bundles of said concentric colored ring coincide.

32. The endoscope according to claim 21, wherein said imaging unit has an imaging medium in a form of a sensor chip of a digital camera.

33. The endoscope according to claim 32, wherein said imaging unit has imaging optics covering a field of view adapted to a size of a projection field.

34. The endoscope according to claim 33, wherein said imaging optics include a convex mirror having a central opening formed therein and a planar mirror, said convex mirror is convexly arched in a direction of said planar mirror and serves to deflect imaging rays onto said planar mirror and said planar mirror in turn serves to deflect the imaging rays into said central opening of said convex mirror.

35. The endoscope according to claim 34, wherein said imaging medium is disposed behind said convex mirror in relation to the viewing direction of the endoscope.

36. The endoscope according to claim 34, further comprising a prism disposed behind said convex mirror in relation to the viewing direction which serves for a further deflection of the imaging rays onto said imaging medium, wherein a surface normal of said imaging medium does not extend parallel to an axis of the endoscope.

37. The endoscope according to claim 34, wherein said planar mirror has an opening formed therein which serves to allow a passage of light rays extending opposite to the viewing direction of the endoscope.

38. The endoscope according to claim 37, wherein the light rays also pass through said central opening in said convex mirror and land on an area close to a center of said imaging medium.

39. The endoscope according to claim 33, wherein said imaging medium is arranged before said imaging optics in relation to the viewing direction of the endoscope.

40. A method for measuring a topography of a surface by an endoscope having a projection unit and an imaging unit, the projection unit and the imaging unit disposed behind one another in relation to an axis of the endoscope, the imaging unit disposed on the axis of the endoscope in a viewing direction of the endoscope before the projection unit, which comprises the step of:
emitting projection rays by the projection unit, the projection rays emerging laterally and radially from an endoscope wall, the projection rays are reflected by a surface to be measured and are depicted as planar on an imaging medium by the imaging unit in the endoscope, the imaging unit being disposed before the projection unit in relation to the axis of the endoscope.
